(19) United States
(12) Patent Application Publication
(10) Pub. No.: US 2009/0196938 A1
(43) Pub. Date: Aug. 6, 2009

(54) USE OF A MATRIX FOR REMOVING C-REACTIVE PROTEIN FROM BIOLOGICAL FLUIDS

(76) Inventor: Birgit Vogt, Berlin (DE)

Correspondence Address: J C PATENTS, INC., 4 VENTURE, SUITE 250, IRVINE, CA, 92618, US

(21) Appl. No.: 12/158,684

(22) PCT Filed: Dec. 22, 2006
(86) PCT No.: PCT/DE06/02335

§ 371 Date: Aug. 25, 2008

(30) Foreign Application Priority Data
Dec. 22, 2005 (DE) 10 2005 061 715.8

(51) Int. Cl. A61K 35/16; A61K 35/14; A61K 35/12; A61K 35/26; A01N 1/02

(52) U.S. Cl. 424/530; 424/529; 424/537; 424/577; 435/2

(57) ABSTRACT

The present invention relates to a method for treating the risk of increased C-reactive protein (CRP) levels by conducting extracorporeal perfusion of blood plasma from patients with risk for cardiovascular diseases or immune dysfunctions, such as autoimmune diseases, through a device, such as a column, which contains absorbent matrix material including lipids, peptides, polypeptides, phosphocholine (PC) or PC derivatives so as to remove C-reactive protein. Moreover, the present invention relates to the use of compounds which have the characteristic to bind CRP at least temporarily, for removing CRP from biological fluids of a patient for prophylaxis and/or treatment of autoimmune diseases, cardiovascular diseases, such as infarction, stroke, diabetes, rheuma and renal failure.

The present invention relates to a method for reducing the risk triggered by increased C-reactive protein (CRP) levels by conducting extracorporeal perfusion of blood plasma from patients with risk for cardiovascular diseases through a device, such as a column, which contains absorbent matrix material including lipids, peptides, polypeptides, phosphorylcholine (PC) or PC derivatives so as to remove C-reactive protein. In particular, the present invention relates to the use of compounds which have the characteristic to bind CRP at least temporarily, for removing CRP from biological fluids of a patient for prophylaxis and/or treatment of immune dysfunctions, autoimmune diseases, cardiovascular diseases, infarction, stroke, diabetes, rheuma and renal failure.

### BACKGROUND OF THE INVENTION

Cardiovascular disease is a major cause of death in the United States and a major source of morbidity, medical cost, and economic loss to millions of people. Two of the most common and destructive aspects of cardiovascular disease are the appearance of arteriosclerosis and thrombolytic events.

In recent years, a great deal of progress has been achieved in the treatment of cardiovascular disease. This progress has been possible not only because of the advancement of therapeutic intervention in the disease mechanisms, but also through the early identification of patients at risk of developing the disease. Indeed, patient risk identification and early treatment are important features of modern medical practice. Over the last twenty years, a variety of factors and clinical parameters have been identified which correlate with either the current state or the future probability of developing cardiovascular disease. Such risk factors may include measurable biochemical or physiological parameters, e.g., serum cholesterol, HDL, LDL, fibrinogen levels, etc., or behavioural of life-style patterns, such as obesity, smoking, etc. The risk factor most germane to the present invention is the level of C-reactive protein (CRP). CRP is induced by IL-6.

The intrinsic relationship between a measurable parameter or risk factor and the disease state is not always clear. In other words, it is not always clear whether the risk factor itself is causative or contributory to the disease or is instead an ancillary reflection that is indicative of the disease. Thus, a therapeutic modality, which affects a risk factor, may be directly modifying a pathological mechanism of the disease or its future course, or may be indirectly benefiting some contributory process related to the disease.

Additionally, many risk factors associated with cardiovascular disease are involved in other pathological states in either a causative or indicative role. Therefore, reduction or blockade of a particular risk factor in cardiovascular disease may have other beneficial effects in other diseases related to that risk factor.

Of particular interest to the methods of the present invention is the reduction of cardiovascular risk factors associated with abnormally high levels of CRP.

CRP is produced by the liver in response to IL-6 production. IL-6 is produced as part of an inflammatory response in the body. Thus, CRP as well as IL-6 levels are markers of systemic inflammatory activity. Chronic inflammation is thought to be one of the underlying and sustaining pathologies in cardiovascular disease.

At menopause, with the loss of estrogen, women's prevalence of cardiovascular disease increases. Also, the risk factors of cardiovascular disease increase, especially lipid (cholesterol and triglyceride), homocysteine, and C-reactive protein (CRP) levels. Today, the most common method of preventing cardiovascular disease in post-menopausal women is Hormone Replacement Therapy (HRT). However, many women do not comply with this therapy because of the unpleasant side-effects, such as bloating, resumption of mensus, breast tenderness, fear of uterine and breast cancer, etc. Additionally, while HRT does lower cholesterol and homocysteine levels, HRT raises CRP and IL-6 levels.

An object of the invention is to provide a therapeutic agent which lowers these risk factors extracorporeally as e.g. by apheresis.

Yeh (Clin Cardiol. 2005 28:408-412) discloses that CRP can be used to predict cardiovascular disease risk, and also that CRP is an indicator of inflammation and that inflammation promotes all stages of atherosclerosis. Zoccali et al., (Semin Nephrol. 2005 25:358-362) discloses that CRP is predictive for cardiovascular disease mortality risk in end stage renal disease patients. Nurmohamed et al., (Neth J. Med. 2005 63:376-381) discloses that CRP is predictive for cardiovascular disease mortality risk in haemodialysis patients. However CRP is only considered as an indicator molecule.

Sola et al., (J Card Fail. 2005 11:607-612) discloses that statin therapy can be used to reduce the levels of CRP and can reduce cardiovascular disease mortality and morbidity. However this therapy is not sufficient to significantly reduce the high level of CRP (up to 1000 times above normal) present after cardiac infarct or the high levels of CRP in dialysis patients.

WO 2004/045596 discloses a method of lowering of plasma CRP levels to reduce systemic inflammation by administering a therapeutic compound. However it does not disclose the extracorporeal treatment of biological fluids for removing CRP from said biological fluids.

JP 2002035117 discloses the use of a column for adsorbing CRP to treat inflammatory disease. However, it also teaches that the column removes inflammatory leukocytes. The removal of leukocytes is not desired in the present invention as this would further stress the patient.

WO 2004/076486 discloses a method for inhibiting immunologic, inflammatory and/or pathophysiological response by administering to patients with increased CRP levels CRP binding molecules. However it does not disclose the extracorporeal treatment of biological fluids for removing CRP from said biological fluids.

Object of the present invention is to provide means and methods for prophylaxis and/or treatment of immune dysfunctions, cardiovascular diseases, infarction, stroke, diabetes, rheuma and renal failure.

This object is solved by the teaching of the independent claims. Further advantageous embodiments result from the description, the examples and the dependent claims.

Surprisingly, the object of the present invention was solved by the at least partial removing of the C-reactive protein from biological fluids, especially from human blood.

Therefore, the present invention provides means, molecules and methods for the extracorporeal treatment of biological fluids, such as human blood and blood plasma, peritoneal fluids and lymphatic fluids which comprise removing C-reactive protein from said biological fluids for improving inflammatory, autoimmune and vascular parameters.

Thus, the present invention relates to the use of a matrix containing compounds which have the characteristic to bind CRP at least temporarily, for removing CRP from biological fluids of a patient for prophylaxis and/or treatment of immune dysfunctions, autoimmune diseases, cardiovascular diseases, such as infarction, diabetes, rheuma, stroke and renal failure.

Another aspect of the present invention is directed to the use of a matrix containing compounds which have the characteristic to bind CRP at least temporarily, for the preparation of a pharmaceutical means for removing CRP from biological fluids of a patient for prophylaxis and/or treatment of immune dysfunctions, cardiovascular diseases, infarction, diabetes, rheuma, stroke and renal failure. With the pharmaceutical means preferably a medical product in form of an adsorbent matrix as is known for dialysis apparatuses is concerned. Thus, the means can be in the form of beads, hollow beads, particles, porous particles, substantially spherical particles, substantially spherical hollow particles, fibers or hollow fibers. These means are preferably used in appropriate apparatuses, such as columns, or dialysis apparatuses for removing CRP of biological fluids of a patient.

With other words the invention relates to the use of compounds which have the characteristic to bind CRP at least temporarily, for the preparation of a pharmaceutical matrix for removing CRP from biological fluids of a patient for prophylaxis and/or treatment of immune dysfunctions, cardiovascular diseases, infarction, stroke, diabetes, rheuma and renal failure.

The immune dysfunctions refer to immune diseases, autoimmune diseases and immune responses to transplanted organs or tissue and are selected from the group comprising or consisting of rejection reactions in transplantations, allo-transplant rejection, xeno-transplant rejection, graft-versus-host rejection, host-versus-graft rejection, diabetes mellitus, rheuma, rheumatoid arthritis, multiple sclerosis, myasthenia gravis, psoriasis vulgaris, Graves' disease, morbus Basedow, Goodpasture syndrome, idiopathic thrombocytopenia purpura (ITP), aplastic anemia, inflammatory bowel disease, morbus Crohn, colitis ulcerosa, dilatative cardiomyopathy (DCM), autoimmune thyroiditis, Hashimoto's thyroiditis, hormone replacement therapy (HRT), osteoarthritis.

The cardiovascular diseases are selected from the group consisting of or comprising infarction, stroke, diabetes, rheuma, renal insufficiency, renal insufficiency due to hypertension, endothelial lesion, endothelial destruction, arteriosclerosis, thrombosis, atherosclerosis, stenosis, restenosis, atherosclerotic or thrombotic diseases, blood flow insufficiency, ischemic events, pulmonary embolism, stable and unstable angina pectoris, coronary arterial diseases, acute cardiac death as well as pathologic results of arteriosclerotic or thrombotic diseases. Especially important and preferred cardiovascular diseases are infarction, stroke, diabetes, rheuma.

Moreover, the cardiovascular diseases comprise especially acute endothelial lesions and acute endothelial destructions as well as long term endothelial lesions (chronic endothelial destructions as well as chronic long term endothelial lesions) and long term endothelial destructions.

The acute endothelial lesions and the acute endothelial destructions represent acute endothelial lesions and acute endothelial destructions preferably after stroke, with blood flow insufficiency, after infarction, after acute cardiac death, after sudden cardiac death, with burns, with surgery lesions or other lesions with partially large lesions areas, with diabetic shock, with acute liver failure, with pancreatitis, with neurodegenerative diseases, with leukaemic patients after irradiation, with coronary arterial diseases or with pulmonary embolism.

The long term endothelial damages and long term endothelial destructions are those preferably with atherosclerosis, with stable and instable angina pectoris, with diabetes mellitus, with rheuma, with rheumatoid arthritis, with multiple sclerosis, with myasthenia gravis, with psoriasis vulgaris, with Graves' disease, with morbus Basedow, with Goodpasture syndrome, with idiopathic thrombocytopenia purpura (ITP), with aplastic anemia, with inflammatory bowel disease, with morbus Crohn, with colitis ulcerosa, with dilatative cardiomyopathy (DCM), with autoimmune thyroiditis, with hashimoto's thyroiditis, with hormone replacement therapies (HRT) as well as with osteoarthritis.

The compounds which have the characteristic to bind CRP at least temporarily are compounds selected from the group comprising or consisting of lipids, lysophospholipids, lysophosphatidylcholines, peptides, peptides with charged amino acids, peptides containing the sequence ArgProArg, polypeptides, antibodies, monoclonal antibodies, antibody fragments, engineered antibodies, phosphocholines, derivatives of phosphocholine, phosphatidylserine, serinkephalines, DNA, DNA derivatives and aptamers.

Preferred are phosphocholines and phosphoethanolamines of the general formula I

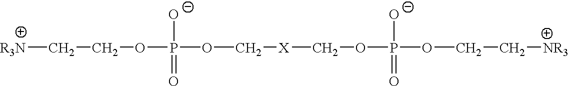

wherein
X is a chemical single bond or an alkyl group with 1 to 20 carbon atoms, preferred 1 to 12 carbon atoms, or an aryl group with 6 to 18 carbon atoms or a cycloalkyl group with 3 to 7 carbon atoms or an alkylcycloalkyl group with 3 to 20 carbon atoms, preferred 3 to 12 carbon atoms or an arylalkyl group with 7 to 20 carbon atoms or an aryldialkyl group with 8 to 20 carbon atoms and
the groups R are independently from each other hydrogen or linear or branched as well as saturated or unsaturated alkyl groups with 1 to 10 carbon atoms as well as enantiomeric forms, racemates, enantiomeric mixtures, diastereomeric mixtures, salts, hydrates, solvates and regioisomers of the afore mentioned compounds.

The alkyl, aryl, cycloalkyl, alkylcycloalkyl, arylalkyl and aryldialkyl groups can be further substituted with one or more functional groups, such as —OH, —OCH₃, —OC₂H₅, —SH, —NO₂, —F, —Cl, —Br, —I, —N₃, —CN, —OCN, —NCO, —SCN, —NCS, —CHO, —COCH₃, —COC₂H₅, —COOH, —COCN, —COOCH₃, —COOC₂H₅, —OOC—CH₃, —OOC—C₂H₅, —CONH₂, —NH₂, —NHCH₃, —NHC₂H₅, —N(CH₃)₂, —N(C₂H₅)₂, —SOCH₃, —SOC₂H₅, —SO₂CH₃, —SO₂C₂H₅, —SO₃H, —SO₃CH₃, —SO₃C₂H₅, —SO₂NH₂, —OCF₃, —OC₂F₅, —O—COOCH₃, —O—COOC₂H₅, —NH—CO—NH₂, —NH—CS—NH₂, —NH—C(═NH)—NH₂, —O—CO—NH₂, —O—CO—OCH₃, —O—CO—OC₂H₅, —CH₂F, —CHF₂, —CF₃, —CH₂Cl, —CH₂Br, —CH₂I, -Ph, —CH₂-Ph, —CH₃, —C₂H₅, —C₃H₇, —CH═CH₂, —CH₂—CH═CH₂, —C≡CH

and/or contain one or more heteroatoms, such as O, S, N, P.

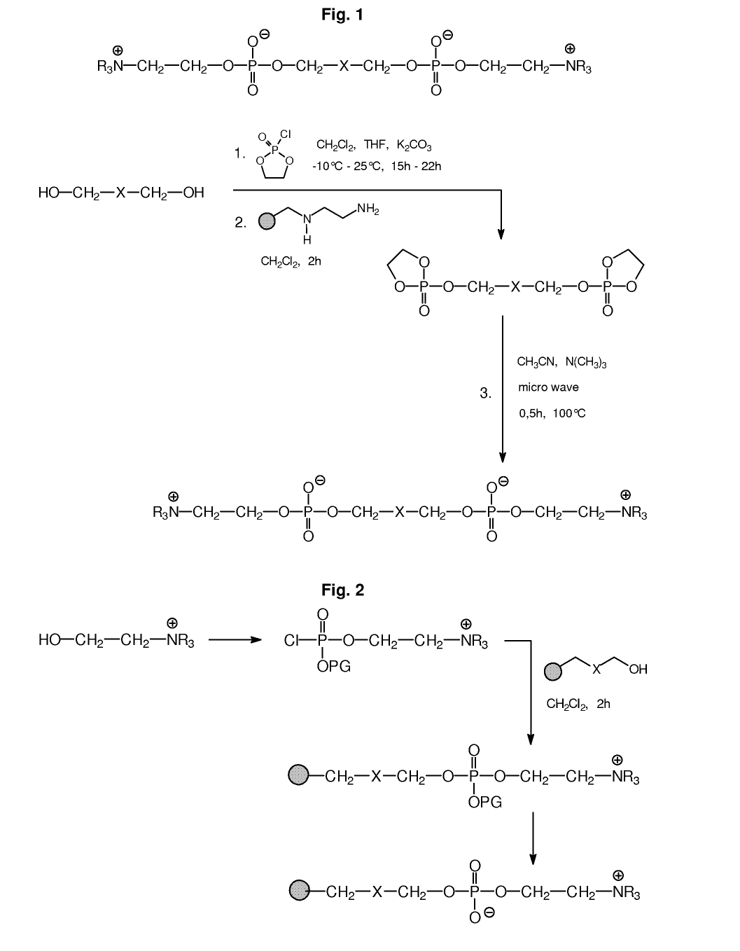

FIG. 1 shows a possible synthesis for such phosphocholines. Explicit examples are the following compounds: 1,5-bis(phosphocholine)pentane, 1,6-bis(phosphocholine)hexane, 1,7-bis(phosphocholine)heptane, 1,4-bis(phosphocholine)di-methylcyclohexane. Besides the bisphosphocholines of course tris-, tetra- and pentaphosphocholines can be used also.

Moreover, especially phosphorylcholines resp. phosphoethanolamines and phosphorylserines of the following general formulas (IIA) and (IIB) are preferred which are bound covalently to a solid carrier:

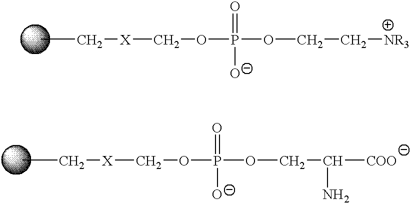

FIG. 2 shows a possible synthesis path for such phosphorylcholines. X is any linker and preferably a linker of the above definition for X. In addition to the above definitions for X said linker can contain the following groups: —O—, —S—, —NH—, —CH═CH—, —C≡C—, —O—(CH₂)ₘ—, —NH—(CH₂)ₚ—, —CO—, —CO—O—, —NH—CO—, —CO—NH—, —O—CO—, —O—CO—O—.

Moreover, glycerophosphorylcholines resp. glycerophosphorylethanolamines as well as glycerophosphorylserines of the general formulas (IIIA), (IIIB), (IIIC), (IIID), (IIIE), (IIIF), (IIIG), (IIIH) are preferred which are bound covalently to a solid carrier:

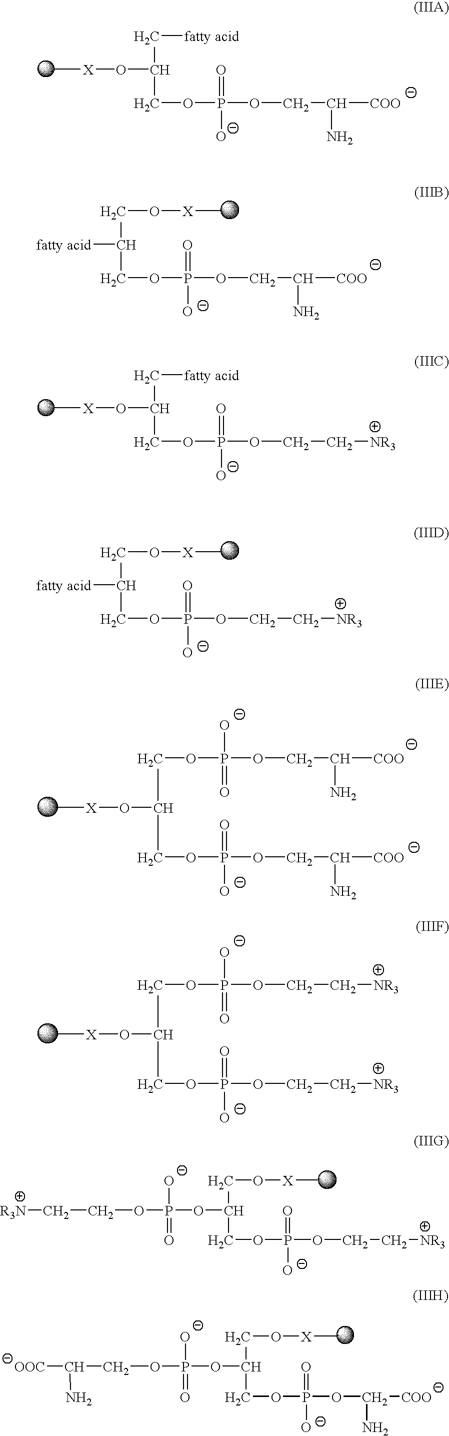

As fatty acids the usual saturated, monoolefinic, polyolefinic, monoacetylenic, unsaturated, linear and/or branched fatty acids with 8 to 28 carbon atoms can be used. Preferred fatty acid groups are palmitoyl, arachidonoyl, oxovaleroyl, glutaroyl, epoxyisoprostan, stearoyl. The fatty acid groups are bound to glycerine via an ester group (COO). Thus, —CH₂-fatty acid means —CH₂—O—CO—R′, wherein R′ represents the carbon chain of the fatty acid.

The mentioned bisphosphocholine compounds, bisphosphoethanolamine compounds, phosphorylcholines, phosphoethanolamines, glycerophosphorylcholines, phosphorylserines, glycerophosphorylethanolamines as well as glycerophosphorylserines can be immobilized ionically to a solid carrier or can be immobilized by means of crosslinking compounds, such as polymers, on the surface of solid carriers. As solid carriers which are also referred to as the pharmaceutical means mentioned herein the herein mentioned particles or fibers or granulates of ceramics, glass, polymers, alumina, silica, silica gel, sepharose, etc. can be mentioned. The solid carriers are illustrated as a circle with dashes in the figures.

These above mentioned compounds are further bound preferably covalently direct or via a linker or ionically or adsorptively to a carrier or are incorporated into a polymer layer on a carrier or are bound to such a polymer layer. Examples of such carriers are matrix forming substances and matrix substrate materials, such as for example eupergit, polysulfone, polyvinylpyrrolidone, formatted silicon, sepharose, acryl beads, agarose, cellulose matrices, ceramic matrices, glass beads and/or solid phase silica or mixtures and/or derivatives of said materials. As matrix, as used herein, matrix substrate materials loaded with compounds which have the characteristic to bind CRP at least temporarily are understood.

In principal, all of the inert chromatography or column materials which especially do not react with blood or blood plasma or alter or contaminate blood resp. blood plasma such that it cannot be injected to a patient after contact with the matrix are suitable for the preparation of the matrix. In principal, all compounds can be considered which can be immobilized to matrix substrate materials and which are capable to bind CRP at least temporarily reversibly or also to bind permanently irreversibly.

Moreover, it was found that it is not always necessary to remove CRP completely, i.e. more than 90%, from the biological fluid. Partially, the therapeutic or prophylactic success is already achieved when the CRP level is reduced to a normal value again.

Thus, the present invention also refers to a method for removing or reducing CRP in biological fluids by contacting the biological fluid with the matrix and the compounds which have the characteristic to bind CRP at least temporarily, bind CRP at least temporarily. After the separation of the biological fluid, such as blood or blood plasma, from the matrix a biological fluid with a reduced or clearly reduced CRP level is obtained which then in turn can be administered via infusion to a patient in need thereof.

The present invention further provides a method and system for removing C-reactive protein from said biological fluids, which comprises pumping blood from a patient through a device which contains adsorbent matrix material that specifically binds CRP and removes CRP from the blood so as to produce treated blood, and returning the treated blood to the same patient, and/or pumping blood from a patient through a cell separator which separates the cells into blood cells and plasma, passing the blood plasma components through a device, such as a column, which contains adsorbent matrix material, such as phosphocholine, for removing C-reactive protein, and recombining the treated plasma and cells before returning the same to the patient.

Further scope of the applicability of the present invention will become apparent from the detailed description given hereinafter. However, it should be understood that the detailed description, while indicating preferred embodiments of the invention, is given by way of illustration only, since various changes and modifications within the spirit and scope of the invention will become apparent to those skilled in the art from this detailed description.

### DESCRIPTION OF THE INVENTION

The object of the present invention is to provide tools, molecules and methods for decreasing levels of CRP in humans. This object is solved by the use of a mixture comprising at least a structural entity which binds CRP or is an antagonist for CRP which mixture depletes CRP from a solution or blocks at least one or more CRP functions on cell surfaces or in a solution for manufacturing of a matrix for extracorporeal treatment of biological fluids for the treatment or prevention of diseases selected from the group consisting of endothelial injury, destruction, increased risk for endothelial injury or destruction or immune disorders and combinations thereof.

Further, the present invention relates to a method for inhibiting conditions or detrimental effects caused by an excess of CRP, wherein said CRP is removed by passage of human blood and blood plasma, peritoneal fluids and lymphatic fluids through a device which contains an adsorbent matrix that specifically binds CRP that includes lipids, preferably lysophosphatidylcholine, peptides, preferably containing charged amino acids especially with an ArgProArg backbone, polypeptides, preferably antibodies, especially monoclonal antibodies, antibody fragments or engineered antibodies, phosphorylcholine (PC) or PC derivatives and DNA or DNA derivatives (e.g. aptamers).

The present invention is based on the finding that molecules that bind CRP, i.e., lipids, preferably lysophospholipids, most preferably lysophosphatidylcholine, peptides, preferably containing charged amino acids, especially with an ArgProArg backbone or with the sequence Gly-Pro-Arg-Pro-Lys, polypeptides, preferably antibodies especially monoclonal antibodies, antibody fragments or engineered antibodies, a recombinant antibody (such as e.g. single chain antibody—scAb or scFv; bispecific antibody, diabody), phosphocholine (PC) or PC derivatives are useful for lowering the levels of CRP.

As used herein, the term “effective amount” means an amount of a mixture or molecule which binds CRP which is capable of decreasing levels of CRP and/or inhibiting conditions or detrimental effects caused by an excess of CRP, respectively.

The term “estrogen deficient” refers to a condition, either naturally occurring or clinically induced, where a woman can not produce sufficient estrogenic hormones to maintain estrogen dependent functions, e.g., menses, homeostasis of bone mass, neuronal function, cardiovascular condition, etc. Such estrogen deficient situations arise from, but are not limited to, menopause and surgical or chemical ovarectomy, including its functional equivalent, e.g., medication with GnRH agonists or antagonists, ICI 182780, and the like.

The term “inhibiting” in the context of inhibiting conditions or detrimental effects caused by an excess of CRP includes its generally accepted meaning, i.e., blocking, prohibiting, restraining, alleviating, ameliorating, slowing, stopping, or reversing the progression or severity of an increase of CRP and the pathological sequelae, i.e. symptoms, resulting from that event.

The term “pharmaceutical” when used herein as an adjective, means substantially non-toxic and substantially non-deleterious to the recipient.

By “pharmaceutical formulation” or “medicament” or “pharmaceutical composition” it is further meant that the carrier, solvent, excipients and salt must be compatible with the active ingredients of the formulation (a composition of at least a molecule, which binds CRP).

The term “solvate” represents an aggregate that comprises one or more molecules of the solute, with one or more molecules of a pharmaceutical solvent, such as water, buffer, physiological salt solution, and the like.

The objects underlying the present invention are in particular accomplished by the use of a composition comprising at least a structural entity which binds CRP or parts of it, preferably human CRP and which composition:
a) inhibits at least one or more CRP functions in a solution, preferably blood or other body fluids, for extracorporeal treatment of biological fluids from patients with acute endothelial injury and/or destruction, preferably for stroke, cardiac infarction, avoidance of sudden cardiac death, for burnt offering, for severe surgery or other injuries with severe wound areas, for diabetic shock, for acute liver failure, neurodegenerative diseases, for leukaemic persons after irradiation and for long term endothelial injury and/or endothelial destruction, especially for patients with atherosclerosis, with unstable angina, with diabetes type I or type II, with excessive body weight and/or obesity, for alcoholics, under Hormone Replacement Therapy (HRT), for old persons, for smokers and for preventing allo-transplant rejection or xeno-transplant rejection and for the induction of allo-transplant or xeno-transplant tolerance or inhibition of T cell activation and for preventing or treatment of autoimmune diseases, autoimmune liver disease and pancreatitis, dialysis patients, renal failure, and/or
b) depletes CRP from a solution, preferably blood or other body fluids or from tissues, for extracorporeal treatment of biological fluids from patients with acute endothelial injury and/or destruction, preferably for stroke, cardiac infarction, avoidance of sudden cardiac death, for burnt offering, for severe surgery or other injuries with severe wound areas, for diabetic shock, for acute liver failure, neurodegenerative diseases, for leukaemic persons after irradiation and for long term endothelial injury and/or destruction, preferably for patients with atherosclerosis, with unstable angina, with diabetes type I or type II, with overweight and/or obesity, for alcoholics, under Hormone Replacement Therapy (HRT), for old persons, for smokers and for preventing allo-transplant rejection or xeno-transplant rejection and for the induction of allo-transplant or xeno-transplant tolerance or inhibition of T cell activation and for preventing or treatment of autoimmune diseases, autoimmune liver disease, dialysis patients, renal failure, and pancreatitis.

In one embodiment the compound of the invention is a polypeptide comprising a binding site to CRP, preferably an antibody containing an antigen-binding site to CRP. The compound of the invention is in particular a poly- or monoclonal antibody comprising an antigen-binding site to CRP.

The monoclonal antibody comprises particularly an antigen-binding site to CRP and is obtainable after immunizing vertebrates, preferably mammals such as mice, rats, guinea pigs, hamsters, monkeys, pigs, goats, chicken, cows, horses and rabbits. The poly- or monoclonal antibody comprising an antigen-binding site to CRP is preferably humanized according to technologies well-known to the skilled person. The compound of the invention can also be prepared by immunizing humanized mice and/or immune defective mice (as e.g. SCID or nude mice) repopulated with vital immune cells (e.g. of human origin; as e.g. SCID-hu mice).

In a further embodiment the antibody of the invention is a recombinant antibody (as e.g. single chain antibody—scAb or scFv; bispecific antibody, diabody etc.) capable of binding to CRP, in particular by containing the antigen-binding site of an antibody which is cross-reactive with CRP. The antibody molecule of the invention is a humanized or human antibody. Subject matter of the invention is also a host cell, preferably a stable host cell, producing the compound of the invention.

A further embodiment of the invention is at least one recombinant vector comprising the nucleotide sequences encoding the binding molecule according to the invention, operably linked to regulating sequences capable of expressing the antibody in a host cell, preferably as a secretory protein.

A subject of the present invention is also a host comprising, preferably stably transgenic, the vector according to the invention, a prokaryotic or eukaryotic cell line producing a recombinant antibody of the invention as well as a eukaryotic organism, most preferably an animal, a plant or a fungus, producing a recombinant antibody according to the invention.

A subject of the invention is also a method of producing a recombinant molecule of the invention capable of binding to the CRP antigen, comprising culturing a host cell and isolating the binding molecule from the culture medium and/or the host cell.

In another embodiment, the present invention is related with a method for inhibiting immunologic, inflammatory and/or pathophysiological responses by treating patients with increased CRP levels with the CRP-binding molecules according to the invention.

Another subject of the present invention is a pharmaceutical composition for reducing the CRP concentration and/or the vacant CRP concentration, containing an amount of the binding molecules according to the invention which is effective for therapy and/or apheresis and/or dialysis and a pharmaceutically acceptable carrier. In addition to these compounds the medicament may comprise anti-inflammatory substances which are selected from the group consisting of molecules which bind phospholipase A2 (PLA2), secretory PLA2 (sPLA2) type II, sPLA2 type IIA, Interleukin-6, Interleukin-1, Interleukin-15 and/or Tumor Necrosis Factor alpha (TNFα), and/or antagonists, CRP binding molecules, anti-IL-1β molecules, PLA2 antagonists, PLA2-binding molecules, complement blockers, at least another binding molecule for treating inflammatory reactions in cardiovascular risk patients like patients with acute coronary syndromes, myocardial infarction, with renal failure, or dialysis patients or combinations thereof.

Still another object of the invention is a method for reducing inflammatory immune and/or pathophysiological responses by reducing the CRP concentration, a method for reducing endothelial injury and/or destruction by reducing the CRP concentration, a method for acute treatments in the case of acute endothelial injury and/or destruction, preferably for stroke, cardiac infarction, avoidance of sudden cardiac death, for burnt offering, for severe surgery or other injuries with severe wound areas, for diabetic shock, for acute liver failure, for pancreatitis, neurodegenerative diseases, for leukaemic persons after irradiation, a method for continuous treatments in case of long term endothelial injury and/or destruction, especially for patients having a medium CRP level with atherosclerosis, with unstable angina, with diabetes type I or type II, with excessive body weight and/or obesity, for alcoholics, under Hormone Replacement Therapy (HRT), for old persons, for smokers, a method for preventing allo-transplant rejection or xeno-transplant rejection, a method for the induction of allo-transplant or xeno-transplant tolerance or inhibition of T cell activation, and a method for preventing or treatment of autoimmune diseases, the methods comprising administering a patient in need of such treatment to an apheresis or dialysis with a therapeutically effective amount of a pharmaceutical composition/matrix according to the invention.

The compound/matrix of the invention can be combined with other molecules, preferably therapeutics for the respective disease or with other anti-inflammatory substances, like e.g. molecules which bind serum cholesterol, HDL, LDL, or other anti-inflammatory molecules like e.g. molecules which bind IL-6 and/or the IL-6 receptor, which bind anti-IL-1-molecules, IL-15 antagonists, PLA2 antagonists, PLA2 binding molecules, complement blockers, C-reactive protein (CRP) antagonists, CRP binding molecules, anti-IL-1β receptor molecules, PLA2 antagonists at least another binding molecule for treating inflammatory reactions in cardiovascular risk patients like patients with acute coronary syndromes, myocardial infarction, with renal failure, or dialysis patients, and/or combinations thereof.

The methods provided by the present invention are useful in both the treatment and prevention of harmful sequelae associated with elevated levels of CRP. Since CRP serum concentration is related to levels and production of cytokines, which are especially produced in inflammatory processes, the methods of the present invention are useful in treating or preventing inflammatory events and sequelae, thereof. Such inflammatory events include, but are not limited to: renal failure, dialysis, arthritis (osteoarthritis), arterial and venous chronic inflammation, autoimmune diseases, e.g., SLE, multiple sclerosis, myasthenia gravis, Graves' disease (morbus Basedow), psoriasis vulgaris, dilated cardiomyopathy, diabetes mellitus, morbus Bechterew, inflammatory bile disease, ulcerative colitis, Crohn's disease, idiopathic thrombocytopenia purpura (ITP), aplastic anemia, idiopathic dilated cardiomyopathy (IDM), autoimmune thyroiditis, Goodpastures' disease and the like.

Methods of the present invention are useful for treating or preventing pathologic sequelae of atherosclerotic or thrombotic disease. Such pathologies include, but are not limited to stroke, circulatory insufficiency, ischemic events, myocardial infarction, pulmonary embolism, stable and unstable angina, coronary artery disease, sudden death syndrome, and the like.

The present invention further contemplates the use of other currently known clinically relevant agents administered to treat the pathological conditions embodied in the present invention in combination with a compound of at least a molecule which binds CRP.

Moreover, the present invention contemplates that the compounds/matrices of at least a molecule which binds CRP are employed in either a treatment or prophylactic modality.

A preferred embodiment of the present invention is where the human to be administered to a compound/matrix of the invention is female, and more preferred is when that human female is oestrogen deficient.

Another preferred embodiment of the present invention is where the condition caused by an abnormally high level of CRP is a cardiovascular disease, especially arteriosclerosis, atherosclerosis, stenosis, restenosis and thrombosis or other acute treatments in case of acute endothelial injury and/or destruction, like stroke, cardiac infarction, sudden cardiac death, burnt offering, severe surgery or other injuries with severe wound areas, diabetic shock, acute liver failure, pancreatitis, neurodegenerative diseases, leukaemic persons after irradiation or long term endothelial injury and/or destruction, like arteriosclerosis, diabetes type I or type II, excessive body weight and/or obesity, alcoholism, Hormone Replacement Therapy (HRT), old persons, smokers.

A particularly preferred embodiment of the present invention is the use of a compound of at least a molecule which binds IL-6 and/or the IL-6 receptor from an oestrogen deficient women, who is receiving oestrogen or HRT, for the reduction of systemic or local inflammation.

Pharmaceutical formulations/matrices can be prepared by procedures known in the art, such as, for example, a compound/matrix with at least one molecule which binds CRP can be formulated with common excipients, solvents, or carriers, and formed into spheres, strings, and the like.

Examples of said adsorbent matrix are matrix substrate materials selected from the group consisting of eupergit, polysulfone, polyvinylpyrollidone, formatted silicon, sepharose, acryloid beads, agarose, cellulose matrices, ceramic matrices, glass beads and/or solid phase silicia or derivatives thereof.

Process for Removing CRP from Biological Fluids

The present invention relates to a method for removing C-reactive protein from biological fluids to improve inflammatory parameters thereof, and specifically to a method for removing C-reactive protein by conducting extracorporeal perfusion of a patient's blood plasma through a phosphorylcholine-matrix adsorption device so as to improve the patients cardiovascular parameters. The CRP-binding matrix can also contain lipids, preferably lysophospholipids, most preferably lysophosphatidylcholine, peptides, preferably containing charged amino acids especially with an ArgProArg backbone, polypeptides, preferably antibodies especially monoclonal antibodies, antibody fragments or engineered antibodies and/or PC and/or PC derivatives, DNA or DNA derivatives (e.g. aptamers).

It is the further object of the present invention to remove C-reactive protein from the blood of patients by extracorporeal perfusion of their blood plasma through a phosphorylcholine matrix adsorption device. The CRP-binding matrix can also contain lipids, preferably lysophospholipids, most preferably lysophosphatidylcholine, peptides, preferably containing charged amino acids especially with an ArgProArg backbone, polypeptides, preferably antibodies especially monoclonal antibodies, antibody fragments or engineered antibodies and/or PC and/or PC derivatives, DNA or DNA derivatives (e.g. aptamers).

It is yet another object of the present invention to provide a system for pumping blood from a patient to a cell separator which separates the blood into cell and plasma components, which comprises the steps of passing the plasma component through a device which contains an adsorbent matrix material including phosphorylcholine so as to remove C-reactive protein thereby producing treated plasma, recombining the treated plasma and cell components to produce treated blood, and returning the treated blood to the patient is also comprised.

It is another further object of the present invention to remove soluble C-reactive protein from the blood of patients by extracorporeal perfusion of their whole blood through a phosphorylcholine matrix adsorption device. The CRP-binding matrix can also contain lipids, preferably lysophospholipids, most preferably lysophosphatidylcholine, peptides, preferably containing charged amino acids especially with an ArgProArg backbone, polypeptides, preferably antibodies especially monoclonal antibodies, antibody fragments or engineered antibodies and/or PC and/or PC derivatives, DNA or DNA derivatives (e.g. aptamers).

It is yet another object of the present invention to provide a system comprising a blood pump for pumping blood from a patient to a column or device connected to said pump which contains an adsorbent matrix material including lipids, preferably lysophospholipids, most preferably lysophosphatidylcholine, peptides, preferably containing charged amino acids especially with an ArgProArg backbone, polypeptides, preferably antibodies especially monoclonal antibodies, antibody fragments or engineered antibodies, and/or PC and/or PC derivatives, DNA or DNA derivatives (e.g. aptamers), so as to remove C-reactive protein thereby producing treated blood, and returning the treated blood to the patient.

In order to perform the methods of the present invention, an extracorporeal device or column containing a matrix with CRP-adsorbent material therein is provided for the extracorporeal treatment of a biological fluid, such as human blood and blood plasma, peritoneal fluids and lymphatic fluids in order to remove CRP therefrom. The treatment may be conducted by continuously removing a patient's blood separating the blood cells therefrom, treating the separated blood in the CRP-adsorbent column or device so as to remove the CRP and mixing and returning the treated plasma and blood cells directly to the patient. Alternatively, after the blood has been removed from the blood cells separated, the blood cells may be directly reinfused into the patient. The separated plasma may be collected, treated in the CRP-adsorbent column, and then returned to the patient as early as possible.

The CRP-adsorbent material contained in the column which is used in the method of the present invention comprises lipids, preferably lysophospholipids, most preferably lysophosphatidylcholine, peptides, preferably containing charged amino acids especially with an ArgProArg backbone, polypeptides, preferably antibodies especially monoclonal antibodies, antibody fragments or engineered antibodies, and/or PC and/or PC derivatives, DNA or DNA derivatives (e.g. aptamers) bonded to a matrix so as to maximize the activity of the PC and/or PC derivative and the binding capacity of the column or device, while minimizing leakage of the PC and/or PC derivative, as well as other substances, from the column during use.

Generally, an effective amount of CRP-binding substances is used in preparing a column. For example, a few milligrams of PC or other CRP-binding substances per gram of matrix material may be used. One example of a non-limiting range is from about 0.06 to 1.6 mg of the substance per gram of column matrix material. In one preferred embodiment, the PC or PC derivatives are cross-linked to amino groups of a formatted silicon matrix so as to be capable of removing CRP to improve inflammatory parameters.

The PC and PC derivatives useful in the method of the present invention include all PC derivatives that sufficiently bind CRP. Examples of PC derivatives include PC esters, such as p-nitrophenyl-6-(O-phosphorylcholine)hydroxy hexanoate and p-aminophenyl phosphorylcholine.

The matrix contained in the extracorporeal device or column used in the method of the present invention may be formed from any material suitable for carrying the CRP-binding substances, such as silicon, agarose, sepharose, acryloid beads, other suitable polymeric substances and matrixes, and solid phase silica. The solid phase silica matrix may comprise virtually any form of particulate silica including amorphous silicas, such as colloidal silica, silica gels, precipitated silicas, and fumed or pyrogenic silicas; microcrystalline silicas such as diatomites; and crystalline silicas such as quartz.

The silica should have a particle size in the range from about 45 to 120 mesh, usually in the range from about 45 to 60 mesh. Other materials useful for forming a matrix as disclosed in U.S. Pat. No. 4,681,870 may also be used in the device or column employed in the method of the present invention. U.S. Pat. No. 4,681,870 is herein incorporated by reference.

The CRP-binding substances are bound to the device or column matrix in a suitable manner so as to retain the ability of the CRP-binding substances to remove CRP from the biological fluids. For example, the CRP-binding substances may be cross-linked to amino groups of a formatted silicon matrix. Other methods for binding PC or PC derivatives to the matrix material may be used such as those applicable methods disclosed by U.S. Pat. No. 4,681,870.

The method of the present invention may be carried out by employing an appropriate extracorporeal device or column containing the CRP-adsorbent matrix material as described above. A removable cartridge may be contained within the column for containing the adsorbent matrix material therein. An example of such a cartridge is described in U.S. Pat. No. 4,681,870. A column or device is connected to a cell separator. The column or device may be sterilized, for example with a gas sterilant such as ethylene oxide, and either used immediately or sealed and stored for later use. Prior to use, the column or device may be washed with normal saline followed by a wash with normal saline containing any other suitable preparatory ingredients. However, no calcium ion chelating agents should be introduced for PC or lysoPC.

The column or device is then connected to the cell separator to receive separated plasma therefrom. The cell separator may be a continuous flow cell separator, or may comprise a semi-permeable membrane which allows passage of the plasma and blood proteins, but prevents passage of the cellular elements of the blood. In the case of a semi-permeable membrane, a blood pump is used to pass the blood through the membrane. Suitable blood pumps include a tube and a peristaltic pump wherein the blood is isolated from the pumping machinery to prevent contamination. The blood passes through the cell separator at a rate which may be in the range of from about 10 to 500 ml/min, typically until a total desired volume of blood has been passed. The blood cells are mixed with the plasma passing through the treatment column or device, and the recombined blood returned to the patient. A micro filter may be provided at the outlet of the treatment column or device to prevent passage of macroscopic particles which might be lost from the column or device.

1. Use of a matrix containing compounds which have the characteristic to specifically bind human CRP at least temporarily, for specifically removing CRP from biological fluids of a patient for prophylaxis and/or treatment of immune dysfunctions and cardiovascular diseases.

2. Use according to claim 1, wherein the compounds which have the characteristic to specifically bind human CRP at least temporarily are selected from the group comprising lipids, lysophospholipids, lysophosphatidylcholines, peptides, peptides with charged amino acids, peptides containing the sequence ArgProArg, polypeptides, antibodies, monoclonal antibodies, antibody fragments, engineered antibodies, phosphocholine, derivatives of phosphocholine, DNA, DNA derivatives and aptamers.

3. Use according to claim 1, wherein the matrix further comprises matrix substrate materials selected from the group consisting of eupergit, polysulfone, polyvinylpyrrolidone, formatted silicon, sepharose, acryl beads, agarose, cellulose matrices, ceramic matrices, glass beads and/or solid phase silica or mixtures and/or derivatives of said materials.

4. Use according to claim 1, wherein the biological fluid is human blood, blood plasma, peritoneal fluid or lymphatic fluid.

5. Use according to claim 1, wherein human CRP is not removed completely from the biological fluid, but is only reduced to a normal value.

6. Use according to claim 1, wherein the cardiovascular diseases are selected from the group comprising infarction, stroke, diabetes, rheuma, renal insufficiency, renal insufficiency due to hypertension, endothelial lesions, endothelial destruction, arteriosclerosis, thrombosis, atherosclerosis, stenosis, restenosis, atherosclerotic or thrombotic diseases, blood flow insufficiency, ischemic events, pulmonary embolism, stable and unstable angina pectoris, coronary arterial diseases, acute cardiac death as well as pathologic results of arteriosclerotic or thrombotic diseases.

7. Use according to claim 1, wherein the immune dysfunctions are selected from the group comprising immune diseases, autoimmune diseases, rejection reactions in transplantations, allo-transplant rejection, xeno-transplant rejection, graft-versus-host rejection, host-versus-graft rejection, diabetes mellitus, rheuma, rheumatoid arthritis, multiple sclerosis, myasthenia gravis, psoriasis vulgaris, Graves' disease, morbus Basedow, Goodpasture syndrome, idiopathic thrombocytopenia purpura (ITP), aplastic anemia, inflammatory bowel disease, morbus Crohn, colitis ulcerosa, dilatative cardiomyopathy (DCM), autoimmune thyroiditis, Hashimoto's thyroiditis, hormone replacement therapy (HRT), osteoarthritis.

8-9. (canceled)

10. Matrix for specifically removing human CRP from biological fluids comprising compounds which have the characteristic to specifically bind human CRP at least temporarily, wherein these compounds are selected from: 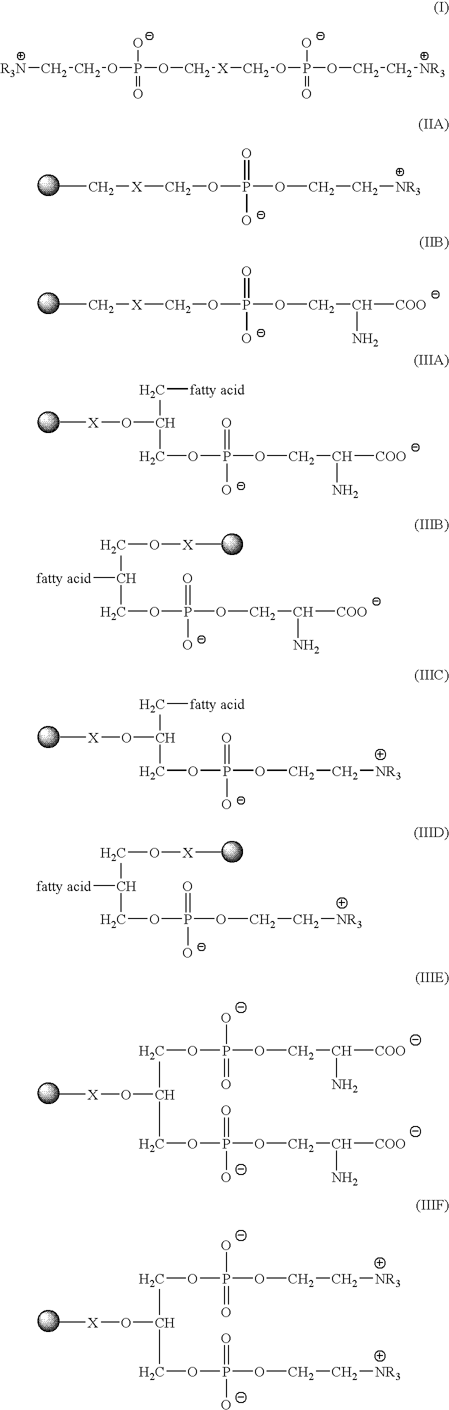 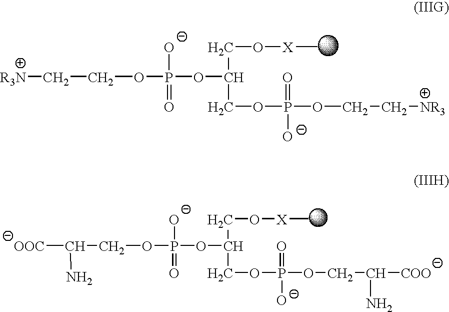
Wherein
X is a chemical single bond or an alkyl group with 1 to 20 carbon atoms or an aryl group with 6 to 18 carbon atoms or a cycloalkyl group with 3 to 7 carbon atoms or an alkylcycloalkyl group with 3 to 20 carbon atoms or an arylalkyl group with 7 to 20 carbon atoms or an aryldialkyl group with 8 to 20 carbon atoms and the group X can contain one or more of the following groups: —O—, —S—, —NH—, —CH═CH—, —C≡C—, 13 O—(CH₂)ₘ—, —NH—(CH₂)ₚ, —CO—, —CO—O—, —NH—CO—, —CO—NH—, —O—CO—, —O—CO—O— and
the groups R are independently from each other hydrogen or linear or branched as well as saturated or unsaturated alkyl groups with 1 to 10 carbon atoms, wherein the alkyl, aryl, cycloalkyl, alkylcycloalkyl, arylalkyl and aryldialkyl groups can be substituted with one or more functional groups selected from the following group: —OH, —OCH₃, —OC₂H₅, —SH, —NO₂, —F, —Cl, —Br, —I, —N₃, —CN, —OCN, —NCO, —SCN, —NCS, —CHO, —COCH₃, —COC₂H₅, —COOH, —COCN, —COOCH₃, —COOC₂H₅, —OOC—CH₃, —OOC—C₂H₅, —CONH₂, —NH₂, —NHCH₃, —NHC₂H₅, —N(CH₃)₂, —N(C₂H₅)₂, —SOCH₃, —SOC₂H₅, —SO₂CH₃, —SO₂C₂H₅, —SO₃H, —SO₃CH₃, —SO₃C₂H₅, —SO₂NH₂, —OCF₃, —OC₂F₅, —O—COOCH₃, —O—COOC₂H₅, —NH—CO—NH₂, —NH—CS—NH₂, —NH—C(═NH)—NH₂, —O—CO—NH₂, —O—CO—OCH₃, —O—CO—OC₂H₅, —CH₂F, —CHF₂, —CF₃, —CH₂Cl, —CH₂Br, —CH₂I, -Ph, —CH₂-Ph, —CH₃, —C₂H₅, —C₃H₇, —CH═CH₂, —CH₂—CH═CH₂, —C≡CH and/or contain one or more heteroatoms selected from the group O, S, N, P, and
the term fatty acids means saturated, monoolefinic, polyolefinic, acetylenic, linear and/or branched fatty acids with 8 to 28 carbon atoms
as well as enantiomeric forms, racemates, enantiomeric mixtures, diastereomeric mixtures, salts, hydrates, solvates and regioisomers of the afore mentioned compounds.
